(19) United States
(12) Patent Application Publication
(10) Pub. No.: US 2008/0208151 A1
(43) Pub. Date: Aug. 28, 2008

(54) MARKER SYSTEM

(75) Inventors: Anne Zacharias, Wexford, PA (US); Robert Frazzini, Wexford, PA (US)

Correspondence Address: THE WEBB LAW FIRM, P.C., 700 KOPPERS BUILDING, 436 SEVENTH AVENUE, PITTSBURGH, PA, 15219, US

(73) Assignee: WHETSTONE MANAGEMENT LLC, Wexford, PA (US)

(21) Appl. No.: 12/034,291

(22) Filed: Feb. 20, 2008

Related U.S. Application Data
(60) Provisional application No. 60/903,080, filed on Feb. 23, 2007.

(51) Int. Cl. A61F 13/15

(52) U.S. Cl. 604/361; 604/358

(57) ABSTRACT

A marker system for providing an indication of the presence of fecal matter, urine, sweat and/or an external fluid source. The system includes at least one reactive material configured to react upon contact with the fecal matter, urine, sweat and/or external fluid source. In this manner, the system provides some unmasked and positive indication of such contact. Also disclosed is a diaper or undergarment.

### CROSS REFERENCE TO RELATED APPLICATIONS

This application claims the benefit of priority from U.S. Provisional Patent Application No. 60/903,080, filed Feb. 23, 2007, which is incorporated herein by reference in its entirety.

### BACKGROUND OF THE INVENTION

1. Field of the Invention

The present invention relates generally to diapers, undergarments and similar articles worn by a user in the genitourinary/anal region and, in particular, to a marker system that is useable to provide a positive signal or indication of the presence of fecal matter, urine, sweat and/or some external fluid source.

2. Description of the Related Art

In the area of care giving, whether to infants, the elderly, those disabled or otherwise unable to care effectively for themselves, one ongoing problem is the attendance required when the person has relieved themselves. Specifically, many persons are unable to communicate that they have relieved themselves in their diapers, undergarments, etc. Accordingly, the caregiver is unaware that this has occurred, and does not realize that the person's diapers or undergarments should be changed as quickly as possible.

If the soiled garments are left in contact with the sensitive skin in the genitourinary/anal region for a lengthy period of time, a rash or ulcers form and other undesired physical conditions may develop, and in certain cases, lead to septic shock. Further, it is desirable to have any wet diaper or undergarment, whether through fecal matter, urine, sweat and/or an external fluid source (e.g., a spilled drink), changed as quickly as possible due to possible irritation in the genitourinary/anal region. In particular, any non-pH balanced material can easily lead to the development of ulcerations or infection, which can lead to other more serious medical conditions.

In order to determine whether the person has, indeed, soiled themselves (or that the diaper or undergarment requires changing), the caregiver usually must visually inspect the article to check for this occurrence. This often results in a difficult or unclean situation for the caregiver. The determination may also be made by the smell emitted from the diaper or undergarment, which would certainly provide an indication that the person has soiled him or herself. However, and in many instances, the odor released from the diaper or undergarment is mild or nonexistent. Therefore, the caregiver would be unaware that the user has relieved themselves in the diaper or undergarment, leading to the above-mentioned deleterious effects.

According to the prior art, various articles and chemical systems have been developed that serve to mitigate, control or mask the odor associated with human bodily fluids. For example, as seen in U.S. Pat. No. 6,369,290 to Glaug et al., a disposable absorbent article is provided. This article includes an odor control powder that is unscented in a dry state, and releases a burst of fragrance when wetted, such as by human waste. Similarly, U.S. Pat. No. 5,733,272 to Brunner et al. also provides compositions and articles that minimize odor caused by body fluids. In particular, a pleasant smell is emitted from the article to indicate that the odor is being removed, and that the product is working. However, none of these systems provide a distinctive marker that acts as both an unmasked indication that the wearer has relieved themselves in their diapers and/or undergarments, as well as provide a positive signal of such an occurrence.

According to the prior art, known diapers include a bottom sheet (moisture-barrier layer), which seeks to inhibit or prevent the flow of liquid from the diaper, and a main, top sheet (wicking layer), which contacts with the baby's or person's skin and allows the liquids to flow into the diaper core or inner area. As is known, the top layer includes a surfactant treatment, which reduces the surface tension of the layer and reduces the contact angle with the liquid, allowing it to pass. Flow dynamics within the diaper core prevent liquids from returning to the surface or wicking layer.

Between the top and bottom layers is an intermediate layer (or inner area), which may be in the form of cellulose or other synthetic fiber. This intermediate layer contains a super-absorbent polymer, such as Aquakeep® and Norsocryl® super-absorbent polymers offered by Arkema AG, for absorption of liquid. Finally, and according to the prior art, a scent inhibiting product, such as sodium bicarbonate or other scent absorbing or masking product, is added to disguise the fact that the diaper has been soiled by the wearer. All of these components are available in the open market and are found in a majority if not all of the diapers available today. The fundamental purpose of these diapers is to retain liquid and conceal, by scent or absorption thereof, the fact that the wearer has soiled the garment.

### SUMMARY OF THE INVENTION

It is, therefore, one object of the present invention to provide a marker system that overcomes the drawbacks and deficiencies of the prior art systems. It is another object of the present invention to provide a marker system that provides an unmasked, unobscured and positive indication to a third-party that a wearer of diapers, undergarments, etc. has “soiled” or otherwise impacted these articles. It is yet another object of the present invention to provide a marker system that provides specified scent and/or visual-based markers that provide an indication of fecal matter, urine, sweat and/or some external fluid source. It is a still further object of the present invention to aid caregivers in making a determination of the state of the wearer's articles without having to physically and visually check within these articles.

Accordingly, provided is a marker system for providing an indication of the presence of fecal matter, urine, sweat and/or an external fluid source. The system includes at least one reactive material configured to react upon contact with the fecal matter, urine, sweat and/or external fluid source. In this manner, the system provides some unmasked and positive indication of such contact.

The present invention is further directed to a diaper or undergarment. The diaper or undergarment includes a marker system for providing an indication of the presence of fecal matter, urine, sweat and/or an external fluid source in the diaper or undergarment. The system includes at least one reactive material configured to react upon contact with the fecal matter, urine, sweat and/or external fluid source, thereby providing some unmasked and positive indication of such contact.

Still further, provided is a marker system for providing an indication of the presence of fecal matter, urine, sweat and/or an external fluid source. The system includes at least one reactive material configured to react upon contact with the fecal matter, urine, sweat and/or external fluid source, thereby providing some unmasked and positive indication of such contact. In addition, the at least one reactive material is formed as a fragrant oil encapsulated within a polymer-based coating layer that at least partially dissolves upon contact with the fecal matter, urine, sweat and/or external fluid source, thereby releasing the fragrant oil from encapsulation.

These and other features and characteristics of the present invention, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular form of “a”, “an”, and “the” include plural referents unless the context clearly dictates otherwise.

### BRIEF DESCRIPTION OF THE DRAWINGS

FIG. 1 is a schematic view of one embodiment of a marker system according to the principles of the present invention;

FIG. 2 is a plan view of another embodiment of a marker system according to the principles of the present invention;

FIG. 3 is a side, sectional view of a further embodiment of a marker system according to the principles of the present invention;

FIG. 4 is a sectional view of another embodiment of a marker system according to the principles of the present invention;

FIG. 5 is a front view of a still further embodiment of a marker system according to the principles of the present invention; and

FIG. 6 is sectional view of another embodiment of a marker system according to the principles of the present invention.

### DETAILED DESCRIPTION OF THE PREFERRED EMBODIMENTS

For purposes of the description hereinafter, the terms “upper”, “lower”, “right”, “left”, “vertical”, “horizontal”, “top”, “bottom”, “lateral”, “longitudinal” and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

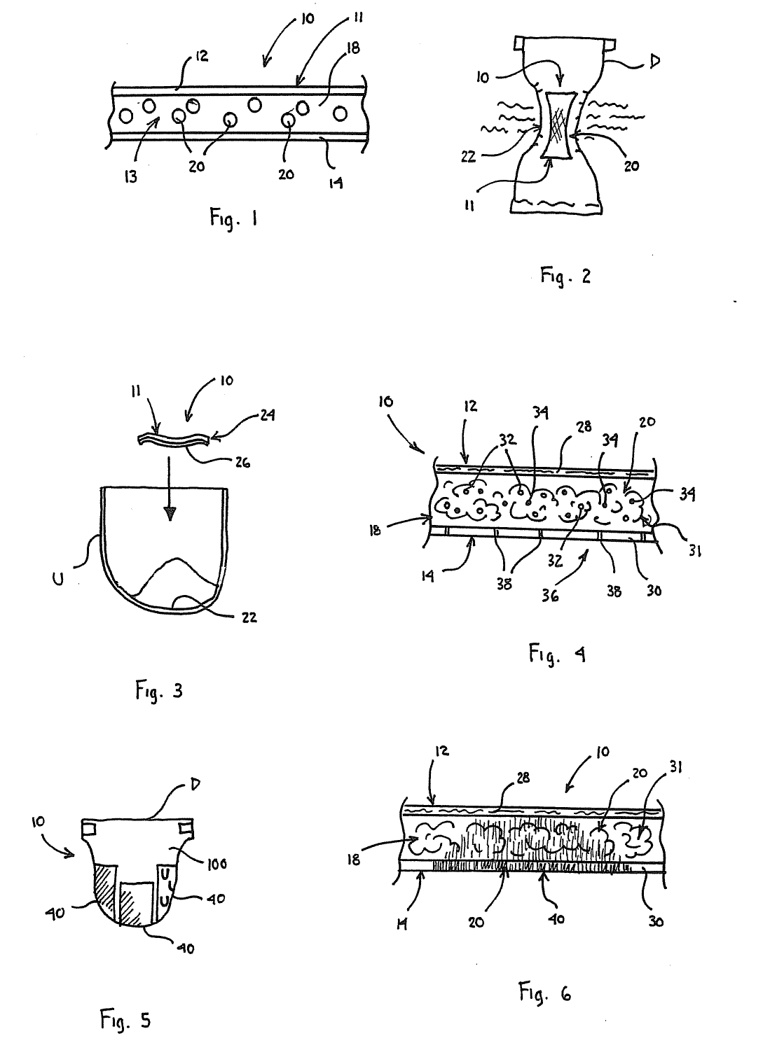

The present invention is directed to a marker system 10 for use in providing a positive and unmasked, unobscured and easily recognized indication of the presence of fecal matter, urine, sweat and/or some external fluid source in an article, such as a diaper D, undergarment U, etc. Of course, the presently-invented system 10 is equally useful in connection with panty liners, incontinence garments, and other articles worn in the person's genitourinary/anal region. The various embodiments of the system 10 of the present invention are illustrated in FIGS. 1-6.

In one preferred and non-limiting embodiment, and as illustrated in FIG. 1, the system 10 includes a containment portion 11 with an inner area 13 that at least partially contains or incorporates some form of a reactive material 20, which, when contacted by fecal matter, urine, sweat and/or some external fluid, reacts. This reaction is dependent upon the specific chemistry and makeup of the reactive material 20, which can be specifically selected to provide an indication of at least one (or any combination) of the following: fecal matter, urine, sweat and/or some external fluid. Upon contact of the material 20 with the fecal matter, urine, sweat and/or some external fluid, some reaction occurs that provides a positive indication of the presence of this foreign material. As discussed hereinafter, this positive indication may be an odor, a visual indication or both, substantially simultaneously.

As also seen in FIG. 1, the containment portion 11 may include an upper layer 12 and a lower layer 14. Together, the upper layer 12 and lower layer 14 form a wall surface 16 which bounds or otherwise contains an intermediate layer 18 (or inner area 13 between the layers 12, 14). Accordingly, it is this intermediate layer 18 that includes or at least partially incorporates the reactive material 20. It is envisioned that at least a portion of the containment portion 11 is in the form of a woven material, a porous material, a mesh material, a layered material, a pouch, a pocket, a single layer, a multi-layer material, an attachable member, a removably attachable member etc.

In another preferred and non-limiting embodiment, the reactive material 20 reacts to wetting of the containment portion 11, with at least some fluid (which, in one preferred embodiment, is water) penetrating the containment portion 11 and contacting the material 20. Therefore, in this embodiment, the reactive material 20 would provide some positive indication of the presence of human fecal matter, human urine, human sweat and/or other external liquid source, such as a drink or other water-based liquid spilled on and penetrating the containment portion 11.

In one preferred and non-limiting embodiment, a specified chemical or reactive material 20 would be embedded or dispersed within a predetermined region 22 of the diaper D or undergarment U. As seen in FIG. 2, this region 22 is substantially coextensive with the wearer's genitourinary/anal region. Further, in this embodiment, the reactive material 20 embedded within the diaper D, when contacted with the fecal matter, urine, sweat and/or some external fluid, releases a scent-based marker or aroma, which would alert the caregiver that the wearer of the diaper D should be changed. It should be noted that the aroma emitted from the diaper D would not be a masking scent, but instead a pleasant scent meant to indicate the presence of urine or feces in the diaper D. For example, the aroma may be a lavender scent or other distinctive odor that is easily recognizable, but pleasant.

It is envisioned that the reactive material 20 may take many different forms. For example, the intermediate layer 18, and/or the reactive material 20 may contain certain chemicals that will react upon contact with the fecal matter, urine, sweat and/or some external fluid, which would serve to emit the alerting aroma. Further, the reactive material may at least partially be in the form of a chemical, a powder, a microencapsulated material, an oil, a fragrance oil, a synthetic material, a natural material, a dye, a material reactive with water, a material reactive with heat, a material reactive with pH, a coated material, a material reactive with a specific, common component of human urine, a material reactive with a specific, common component of human fecal matter, a material reactive with a specified component of a fluid material, etc. Still further, this reactive material 20 may be either manufactured integrally with the diaper D or undergarment U, or applied to the diaper D or undergarment U after manufacturing.

In one preferred and non-limiting embodiment, the reactive material 20 is a fragrant oil encapsulated within a coating layer that at least partially dissolves upon contact with fecal matter, urine, sweat and/or some external fluid, thereby releasing the fragrant oil from encapsulation. For example, the fragrant oil may be fragrant oil encapsulated in a multi-component polymer alloy. Such polymers may range in active fragrance loads from about 15% to about 45%, and these scents may be further processed and encapsulated with a polymer that rapidly breaks down when contacted with or exposed to water, which is normally a common component in fecal matter, urine, sweat and an external fluid.

In another example and embodiment, the polymer encapsulation of the scent elements (oils) can be completed by coating these scent elements with a mixture of saccharide and polymer coating ingredients, thereby forming particulates. Next, these particulates are mixed with water to form a coating solution, e.g., see U.S. Pat. No. 3,953,326 to Groppenbacher et al. Other known approaches can be used to coat the scent elements. For example, one such coating approach uses the method of melt-spinning the above-referenced mixture with water to form a coating solution, and applying the aqueous solution to the scent elements. In one preferred embodiment, the reactive material 20 includes the polymer layer for encapsulating the scent elements, and this polymer layer or coating is between about 5% and about 15% of the overall weight.

In another preferred and non-limiting embodiment, the containment portion 11 is in the form of a removable member 24, which may be placed into and removed from an undergarment U or diaper D. For example, as seen in FIG. 3, the removable member 24 includes an adhesive layer 26, which is applied to or contacted with the appropriate portion or region 22 of the undergarment U. The operation and application of this removable member 24 may be likened to the use of a sanitary napkin or the like. Again, the removable member 24 may come in various dimensions, sizes and shapes so as to provide maximum coverage to the genitourinary/anal region of the wearer, as well as the diaper D or undergarment U. In this manner, if the wearer does not “soil” his or her undergarments U, the removable member 24 may simply be removed and applied to a fresh undergarment U for further use. Similarly, if the wearer does indeed use the removable member 24, it is easily removable from the undergarment U, thereby allowing the caregiver to wash or otherwise dispose of the undergarment U or diaper D.

Yet another preferred and non-limiting embodiment is illustrated in FIG. 4. In this embodiment, the upper layer 12 is a wicking or soft layer 28, which serves as a layer that comes in contact with the genitourinary/anal region of the wearer. This wicking layer 28 would be penetratable by the fecal matter, urine, sweat and/or external fluid source, such that it may appropriately contact the reactive material 20 in the intermediate layer 18 (or inner layer 13). In addition, this wicking layer 28 is formed of a material that also serves to contain or otherwise ensure that the reactive material 20 does not contact the genitourinary/anal region of the wearer.

Further, and in this embodiment, the lower layer 14 is a moisture-barrier layer 30. This moisture-barrier layer 30 would prevent the fecal matter, urine, sweat and/or external fluid source from exiting the diaper D or undergarment U. Accordingly, the intermediate layer 18 (whether comprising, including or incorporating the reactive material 20) would be positioned between the wicking layer 28 and the moisture-barrier layer 30. This intermediate layer 18 may be in the form of an absorbent material 31, such as cellulose, synthetic material, a material treated with a super-absorbent polymer, etc. The reactive material 20 could be suspended in, dispersed throughout, incorporated in or otherwise associated with the absorbent material 31 in the intermediate layer 18.

Also as seen in the embodiment of FIG. 4, a first reactive material 32 and a second reactive material 34 are used. When the marker system 10 is a scent-based marker system, the first reactive material 32 would cause a first scent to be emitted, and the second reactive material 34 would cause a second, different scent to be emitted. In addition, the first reactive material 32 may react only with fecal matter, while the second reactive material 34 would react only with urine. In this manner, the caregiver would understand whether the diaper D or undergarment U has been soaked with urine or contains fecal matter. For example, a diaper D soaked with urine may smell like lavender, while a diaper D soaked with feces may smell like roses. Of course, any number of reactive materials 20 could be selected to emit a unique aroma based upon some unique component found in the fecal matter, urine, sweat and/or external liquid source, thus allowing specific identification of what has occurred in connection the diaper D. This approach may also be effective with visual markers.

In another embodiment, and as illustrated in FIG. 4, the system 10 may include a venting arrangement 36. For example, this venting arrangement may include one or more vent holes 38 extending through a layer 12, 14 of the diaper D or undergarment U. For example, in one preferred embodiment, the vent holes 38 extend through the lower layer 14, which is considered the moisture-barrier layer 30 or “outside” layer of the system 10 or diaper D. Further, these vent holes 38 may be sized and shaped so as to prevent any moisture or material from exiting the layer 12, 14, while allowing the aroma or scent to be emitted from the reactive material 20 and pass through the vent holes 38.

It is further envisioned that, in order to prevent moisture from exiting these vent holes 38, some filter arrangement or other similar barrier could be used, where the barrier allows scent or aroma to pass through the vent holes 38, while preventing any moisture, dampness or other flowable material to pass therethrough. Accordingly, the aroma is not trapped within the diaper D or undergarment U, and instead allowed to pass through the layer 12, 14 and permeate the surrounding area of the diaper D or undergarment U. Therefore, this arrangement allows for a positive indication of the presence of fecal matter, urine, sweat and/or external liquid source in the diaper D or undergarment U, without requiring the caregiver to physically check or be in close proximity.

While, as discussed above, the marker system 10 may be a scent-based marker, it is also envisioned that the marker system 10 is a visual-based marker system. Accordingly, the diaper D or undergarment U may include some area or region that provides a visual indication of the state of the diaper D or undergarment U. For example, in one embodiment, the diaper D or undergarment U may change color to alert the caregiver, upon visual inspection, that it has been soiled with fecal matter, soaked with urine, sweat, external liquid, etc. In addition, as discussed above in connection with the scent-based marker, the diaper D or undergarment U may change one color when soiled with fecal matter, and another color when soiled with urine, such as by using the different reactive materials 32, 34. Still further, the different colors may be combined with different scents. For example, a diaper that smells lavender and has turned blue may be soiled with urine, while a diaper D that smells like roses and has turned green may be soiled with feces.

As shown in the preferred and non-limiting embodiment of FIG. 5, the diaper D or undergarment U may include certain reactive regions or layers 40 positioned on the outside of the diaper D or undergarment U and in the genitourinary/anal region. For example, FIG. 5 illustrates multiple layers 40 positioned on an outer surface 100 of the diaper D. These layers 40 contain the appropriate chemicals or treatments that would allow them to change color in the same process described above in connection with the scent-based marker. In operation, and in using this visual-based marker system 10, the layers 40 may be integrally formed with the diaper D or undergarment U, with the upper layer 12 (wicking layer 28) in contact with the genitourinary/anal region of the wearer, the intermediate layer 18 including the reactive material 20, and the lower layer 14 (moisture-barrier layer 30) acting as the layer 40 that is viewable by a third party.

In one preferred and non-limiting embodiment of the visually-based marker system 10, and as illustrated in FIG. 6, the above-discussed wicking layer 28, intermediate layer 18 and reactive material 20 is utilized. However, in this embodiment, the reactive material 20, when coming into contact with the fecal matter, urine, sweat and/or external liquid source, changes color or otherwise releases a material, e.g., a dye or similar benign material, that contacts with, penetrates, reacts with or otherwise causes the lower layer 14 to change colors, or otherwise provides some visual indication to a third party. Of course, the reactive material 20, in this embodiment, may be positioned in the lower layer 14, thus avoiding the need for multiple layers. In any case, it is the provision of a visual marker to the caregiver that leads to one benefit of the present invention.

It is further envisioned that the reactive material 20 that causes the color change may be a chemical process that either causes the creation or release of a dye, or initiates a chemical reaction that causes the creation of the visual marker. In yet another embodiment of the present invention, the reactive material 20 is activated by the heat of the fecal matter, urine, sweat and/or external liquid source. Accordingly, it would be the heat that initiates the reaction and creation of the visual indication (or, in the scent-based marker system, the aroma), thus providing the caregiver with an understanding of the situation.

Any number of layers and arrangements are envisioned that provide the scent-based marker or the visual-based marker, or any combination thereof. For example, with respect to the visual-based marker, the diaper D or undergarment U may be created of one or more layers, where the outermost layer reacts with either the chemical makeup of the fecal matter, urine, sweat and/or external liquid source, or the heat of the fecal matter, urine, sweat and/or external liquid source, which serves to initiate the creation of this marker. For example, the reactive material 20 may be an ink, dye or other material that changes color or strengthens color based upon the reaction.

Accordingly, the diaper D or undergarment U may include the layers 40 in the form of or marked with a reactive “U” or “1”, which would indicate that the diaper D or undergarment U has been soiled with urine, and a reactive “F” or “2”, which would indicate that the diaper D or undergarment U has been soiled with fecal matter. Other text, symbols, etc. could be used to indicate the presence of sweat, some specified external liquid source, juice, etc. Therefore, the caregiver need only look at the diaper D or undergarment U to easily establish this occurrence. Again, this visual-based marker may also be combined with the scent-based marker discussed above. This combination would allow the scent-based marker to gain the caregiver's attention, and the visual-based marker to establish what has occurred with respect to the wearer.

As seen in FIG. 5, the layers 40 provide some visual indication to the caregiver regarding the state of diaper D or undergarment U. It is also envisioned that, as with the embodiment of FIG. 3, the layers 40 are applicable to the outer surface 100 of the diaper D or undergarment U. Accordingly, the layers 40 may include the above-discussed adhesive layer 26, allowing them to be attached to the outer surface 100 of the diaper D or undergarment U. Therefore, these layers 40 could be removable and reused if not reacted in a previous instance.

In addition, the layers 40 could be positioned at specified locations on the outer surface 100 of the diaper D or undergarment U. Still further, the layers 40 could be sold as a strip of material that allows the caregiver to easily position them in the desired location on the diaper D or undergarment U. Also, as discussed, these layers 40 may be made of the upper layer 12, lower layer 14 and intermediate layer 18 (having the reactive material 20 therein), in the form of a pocket, a strip, a bounded area, etc. Therefore, these layers 40 may be in the form of a peel-off strip of material that can be cut to the size and shape desired.

Any number of embodiments and arrangements is envisioned that provide the appropriate markers and indications to the caregiver about the state of the diaper D or undergarment U. In addition, in the case of the scent-based marker, the alerting aroma may be a pleasant smell, such as lavender or roses, which will allow the wearer to smell a soothing scent that may calm them until the diaper D or undergarment U is changed. In addition, by using a diaper D or undergarment U with an alerting aroma built in, the caregiver will change the diaper D more often possibly resulting in fewer cases of diaper rash or other medical conditions that may arise from a response to the soiling. In addition, by using the visual-based marker, the caregiver is easily able to ascertain the state of the diaper D or undergarment U when inspected. It is further envisioned that the aroma provided in the scent-based marker could have therapeutic and other positive benefits with respect to the wearer. By using the visual-based marker, the caregiver can immediately understand and recognize what type of “soiling” has occurred and react accordingly, without having to inspect the inside of the diaper D or undergarment U. The presently-invented system 10 can be incorporated with, attached to, removed from or manufactured integrally with a diaper D or undergarment U.

In this manner, the present invention provides a marker system 10 that, as opposed to masking or obscuring the scent, provides some positive indication of the presence of fecal matter, urine, sweat and/or external liquid source in the wearer's diaper D or undergarment U. The present invention intends not to inhibit scent and thus mask the fact that soiling has occurred, and instead provides a positive indication of the presence of certain materials. For example, provided is a system 10 having an intermediate layer 18 or inner area 13 that includes (distributed within the cellulose layer) a water-reactive polymer-coated, microencapsulated fragrance that releases scent over time. Once wetted or contacted, this scent is strong enough to alert a caregiver who comes within proximity of the wearer, but not strong enough as to overwhelm the wearer. Accordingly, in the present invention scent is used to alert and communicate the state of the diaper D or undergarment U. The specific amount of scent elements distributed or utilized may vary by purpose, i.e., infant diapers D and undergarments U require less scent than adult diapers D and undergarments U, and type of scent, e.g., lavender requires a denser scent than peppermint.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A marker system for providing an indication of the presence of at least one of the following: fecal matter, urine, sweat, an external fluid source or any combination thereof, the system comprising at least one reactive material configured to react upon contact with the fecal matter, urine, sweat and/or external fluid source, thereby providing some unmasked and positive indication of such contact.

2. The system of claim 1, further comprising a containment portion having an inner area configured to at least partially contain or incorporate the reactive material therein.

3. The system of claim 2, wherein the containment portion comprises: an upper layer and a lower layer forming wall surface; and wherein the inner area at least partially comprises an intermediate layer positioned substantially between the upper and lower layers, wherein the intermediate layer at least partially comprises the reactive material.

4. The system of claim 2, wherein the containment portion is in the form of a removable member configured to be associated with and removed from at least one of the following: a diaper, an undergarment, a panty liner, an incontinence garment, a garment in contact with a person's genitourinary/anal region.

5. The system of claim 2, wherein the unmasked and positive indication is a scent, and wherein the containment portion comprises at least one venting arrangement associated therewith and configured to permit the scent to be emitted from the system to the surrounding area.

6. The system of claim 2, wherein at least a portion of the containment portion is in the form of at least one of the following: a woven material, a porous material, a mesh material, a layered material, a pouch, a pocket, a single layer, a multi-layer material, an attachable member, a removably attachable member.

7. The system of claim 2, wherein the containment portion includes at least one surface having an adhesive material at least partially positioned thereon, such that the containment portion is removably attachable to a surface of at least one of the following: a diaper, an undergarment, a panty liner, an incontinence garment, a garment in contact with a person's genitourinary/anal region.

8. The system of claim 1, wherein the unmasked and positive indication is a visual indication, and wherein the system further comprises at least one surface visible from a position remote from the system.

9. The system of claim 1, wherein the reactive material at least partially comprises at least one of the following: a chemical, a powder, a microencapsulated material, an oil, a fragrance oil, a synthetic material, a natural material, a dye, a material reactive with water, a material reactive with heat, a material reactive with pH, a coated material, a material reactive with a specific, common component of human urine, a material reactive with a specific, common component of human fecal matter, a material reactive with a specified component of a fluid material.

10. The system of claim 1, wherein the unmasked and positive indication is at least one of the following: a scent, an odor, a smell, an aroma, a visual indication, a color, a pattern, a symbol, text, a phrase, a word, a visual representation of an object.

11. The system of claim 1, further comprising:
a first reactive material configured to provide a first unmasked and positive indication of contact with at least one of the following: fecal matter, urine, sweat, external liquid source; and
at least one additional reactive material configured to provide a second unmasked and positive indication of contact with another of the following: fecal matter, urine, sweat, external liquid source.

12. The system of claim 1, wherein the first unmasked and positive indication is at least one of the following: a scent, an odor, a smell, an aroma; and the second unmasked and positive indication is at least one of the following: a visual indication, a color, a pattern, a symbol, text, a phrase, a word, a visual representation of an object.

13. The system of claim 1, wherein the marker system is at least partially incorporated into at least one of the following: a diaper, an undergarment, a panty liner, an incontinence garment, a garment in contact with a person's genitourinary/anal region.

14. The system of claim 1, wherein the marker system is in the form of a strip of material attachable to a surface of at least one of the following: a diaper, an undergarment, a panty liner, an incontinence garment, a garment in contact with a person's genitourinary/anal region.

15. The system of claim 1, wherein the reactive material is fragrant oil encapsulated within a coating layer that at least partially dissolves upon contact with the fecal matter, urine, sweat and/or external fluid source, thereby releasing the fragrant oil from encapsulation.

16. A diaper or undergarment, comprising a marker system for providing an indication of the presence of at least one of the following: fecal matter, urine, sweat, an external fluid source or any combination thereof, in the diaper or undergarment, the system comprising at least one reactive material configured to react upon contact with the fecal matter, urine, sweat and/or external fluid source, thereby providing some unmasked and positive indication of such contact.

17. The diaper or undergarment of claim 16, further comprising at least one reactive region associated with an outside surface of the diaper or undergarment, the at least one reactive region at least partially incorporating the at least one reactive material.

18. The diaper or undergarment of claim 16, further comprising:
a wicking layer configured to permit the fecal matter, urine, sweat and/or external fluid source to at least partially contact the at least one reactive material;
a moisture-barrier layer configured to prevent passage of the fecal matter, urine, sweat and/or external fluid source; and
an intermediate layer positioned substantially between the wicking layer and moisture barrier layer, wherein the intermediate layer at least partially comprises the reactive material.

19. The diaper or undergarment of claim 16, wherein the intermediate layer at least partially comprises a fibrous material configured to absorb fluid, the at least one reactive material at least partially associated with the fibrous material.

20. The diaper or undergarment of claim 19, wherein the fibrous material is at least one of the following: a cellulose material, an absorbent material, a synthetic material, a super-absorbent polymer material or any combination thereof.

21. A marker system for providing an indication of the presence of at least one of the following: fecal matter, urine, sweat, an external fluid source or any combination thereof, the system comprising at least one reactive material configured to react upon contact with the fecal matter, urine, sweat and/or external fluid source, thereby providing some unmasked and positive indication of such contact, wherein the at least one reactive material comprises a fragrant oil encapsulated within a polymer-based coating layer that at least partially dissolves upon contact with the fecal matter, urine, sweat and/or external fluid source, thereby releasing the fragrant oil from encapsulation.
